(19) United States
(12) Patent Application Publication
(10) Pub. No.: US 2007/0149899 A1
(43) Pub. Date: Jun. 28, 2007

(54) Inclination measuring device

(75) Inventors: Adi Shechtman, Nofit (IL); Orna Filo, Zurit (IL)

Correspondence Address: DANIEL J SWIRSKY, 55 REUVEN ST., BEIT SHEMESH, 99544, IL

(73) Assignees: Orna Filo, Zurit (IL); ORTHOSCAN TECHNOLOGIES LTD., Yokneam Eilit (IL)

(21) Appl. No.: 10/593,581

(22) PCT Filed: Mar. 6, 2005
(86) PCT No.: PCT/IL05/00259

§ 371 Date: Jan. 31, 2007

Related U.S. Application Data
(60) Provisional application No. 60/549,930, filed on Mar. 5, 2004.

(51) Int. Cl. A61B 5/103; A61B 5/117

(52) U.S. Cl. 600/587; 600/595

(57) ABSTRACT

An inclination measuring device (10) is provided, which includes an inclination tracking device (12) configured to pass over the object, having a plurality of elements, whose angle of inclination is to be mapped and a sensor probe (14) in communication with the inclination tracking device (12). The sensor probe (14) is configured to sense the position of each of the plurality of elements. The inclination measuring device (10) is useful for measuring the angle of trunk inclination or rotation of a person's trunk.

### FIELD OF THE INVENTION

The present invention relates to an inclination measuring device, and more particularly to an inclination tracking device including means for mapping the degree of inclination and rotation of an object.

### BACKGROUND OF THE INVENTION

It is often desirable to measure the degree of inclination of the object. It is especially important in the medical field, for example in measuring the degree of trunk inclination in patients having scoliosis. In order to avoid the over-referral of patients who may have scoliosis, it is common to use a scoliosis screening device (commonly known as a “Scoliometer”), such as that described in U.S. Pat. No. 5,181,525, to determine the angle of trunk rotation. The angle of trunk rotation is defined as: “the angle between the horizontal and a plane across the posterior trunk at the point or points of maximum deformity.”

Scoliosis refers to a lateral spinal curve of a certain degree that affects a large number of people. A contour mapping system and method for mapping the contour of an object such as a person's spine is described in U.S. Pat. No. 6,524,260 assigned to the applicants of the present invention. U.S. Pat. No. 6,524,260 describes a diagnostic system and method which avoids the risk of radiation exposure and which is capable of providing more detailed information about the examined spine, such as the degree of curvature of the spine, or the degree of rotation of any particular vertebra therein. The mapping of the curvature of a person's spine enables the presence and severity of a deformation in the spine, such as scoliosis or Kyphosis to be detected.

### SUMMARY OF THE INVENTION

The applicants have realized that it would be advantageous to be able to automatically or semi-automatically measure the angle of inclination of an object and the degree of rotation of a deformity of a person's back, for example. The applicants have further realized that it would be advantageous to be able utilize a contour mapping system to map and graphical output the results of the measurement of the angle of inclination. A contour mapping system is described in U.S. Pat. Nos. 6,500,131 and 6,524,260, assigned to the applicants of the present invention, the contents of which are incorporated herein.

The present invention provides an inclination measuring device which tracks and measures an object, having a plurality of interconnecting elements, for the purpose of mapping the angle of inclination of the various elements.

In an embodiment of the invention, the inclination measuring device may be configured to measure the angle of trunk inclination or degree of rotation of a deformity of a person's back.

There is thus provided, an inclination measuring device constructed and operative in accordance with an embodiment of the present invention, which includes an inclination tracking device configured to pass over the object whose angle of inclination is to be mapped and a sensor probe in communication with the inclination tracking device. The object may have a plurality of elements and the sensor probe may be configured to sense the position of each of the plurality of elements.

Furthermore, in accordance with an embodiment of the invention, the object to be mapped may be the spine of a person and the elements are the vertebrae of the spine.

Furthermore, in accordance with an embodiment of the invention, the sensor probe may be fixed in relation to the inclination tracking device. The sensor probe may include optical sensors.

Furthermore, in accordance with an embodiment of the invention, the sensor probe may be sensor probe is configured to be removable from the inclination tracking device and may be configured to be attachable to at least one finger of a user's hand.

Furthermore, in accordance with an embodiment of the invention, the sensor probe may further include a position sensor and tracking system in communication therewith.

Furthermore, in accordance with an embodiment of the invention, the inclination tracking device may include one of a group of devices for calculating the angles of inclination including a gyroscopic inclinometer device, inclinometer, accelerometer, a magnetic field generator and Optical 3D tracking systems.

Furthermore, in accordance with an embodiment of the invention, the inclination tracking device may include a processing unit and at least one of a group of devices including a data storage device and a display screen in communication with the processing unit. Additionally, the inclination tracking device may further include a transmitting device for transmitting data to an external source. Furthermore, the inclination tracking device may include an inductor in communication with the processing unit for supplying power via a wireless connection to a unit for recharging the inclination measuring device.

Furthermore, in accordance with an embodiment of the invention, the sensor probe may be configured to record at least one reading for each vertebrae.

Furthermore, in accordance with an embodiment of the invention, the data processor may be programmed to record data including the maximal trunk rotation measurements of at least one of group of vertebrae, including the upper thoracic, mid-thoracic, and lumbar regions of the spine.

Additionally, the processing unit may be programmed to compute and display the data showing at least one of a group including Coronal, Sagittal and Apical views of the spine. Also, the processing unit may be programmed to compute and display the maximum inclination and/or location of the vertebrae in each of the upper thoracic, lower thoracic and lumbar regions of the spine.

Furthermore, in accordance with an embodiment of the invention, the sensor probe may be configured to record at least one of a group includes the vertebral level of the trunk rotation measurements, the direction of inclination of each vertebrae, the difference in height between left and right of each vertebrae and the length of the spine.

Furthermore, in accordance with an embodiment of the invention, the inclination measuring device may be configured to measure the angular deviation irrespective of the position of object being measured.

Furthermore, in accordance with an embodiment of the invention, the inclination tracking device may includes a substantially rectangular housing having an indentation formed in the center of one edge of the housing.

Furthermore, in accordance with an embodiment of the invention, the inclination tracking device includes a pair of tracking devices attached on either side of the indentation, along the bottom edge of the rectangular housing.

Additionally, in accordance with an embodiment of the invention, the inclination tracking device may include markers configured to be used in conjunction with the Optical 3D tracking systems to identify and calculate inclination angles of the vertebrae.

### BRIEF DESCRIPTION OF THE DRAWINGS

For a better understanding, the invention will now be described, by way of example only, with reference to the accompanying drawings in which like numerals designate like components throughout the application, and in which:

FIG. 1 is a schematic illustration of a stand alone inclination measuring device, constructed and operative in accordance with an embodiment of the present invention;

FIG. 1A is a schematic block diagram illustration of the inertial sensors used with the stand alone inclination measuring device of FIG. 1;

FIG. 2 is a schematic block diagram illustration of the internal components of the inclination measuring device of FIG. 1;

FIG. 3 is a schematic illustration of an inclination measuring device, constructed and operative in accordance with a further embodiment of the present invention;

FIG. 4 is a view of the vertebrae of a spine;

FIG. 5 is a side elevational view of a patient, whose angle of trunk inclination is being measured; and

FIG. 6 is a graphical illustration of the results of the measurements of a trunk rotation, using the device of FIG. 1.

### DESCRIPTION OF THE PREFERRED EMBODIMENTS

In one embodiment of the invention, a non-invasive, automatic or semi-automatic system and method to measure the degree of rotation of a deformity of the back found on routine spinal examination is provided.

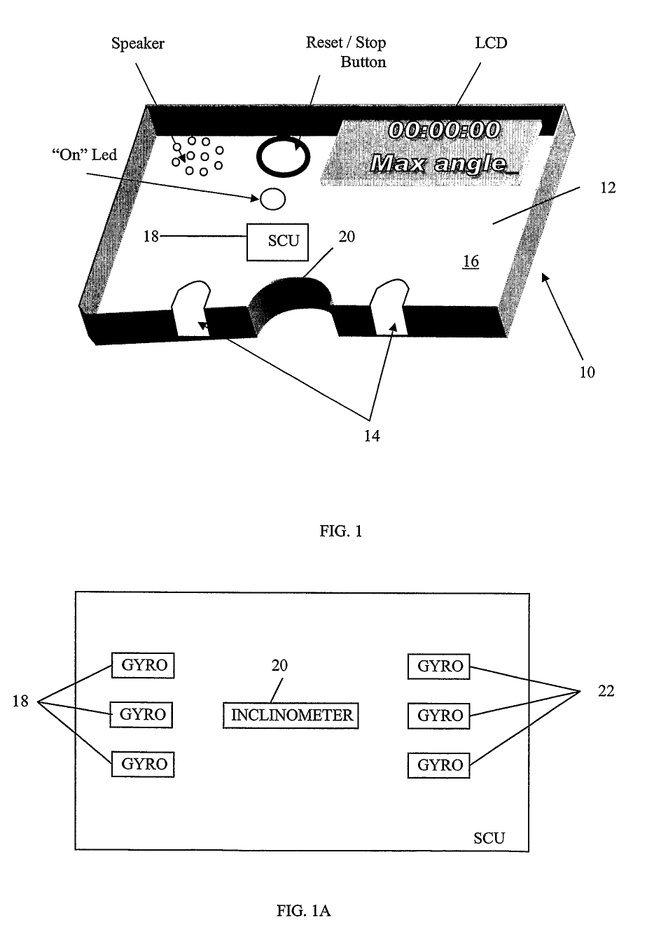

Reference is now made to FIG. 1, which is a schematic illustration of a stand alone inclinometer (or inclination measuring device), generally designated 10, which is constructed and operative in accordance with an embodiment of the present invention. The inclinometer 10 comprises an inclination tracking device 12 and sensor probes 14.

The inclination tracking device 12 houses a sensing and computing Unit (SCU) 16, which comprises inertial sensors and electronic components, embedded into the inclinometer. In an embodiment of the invention, shown in FIG. 1A, to which reference is also made, the inertial sensors may include a plurality of gyros 18, a two-axis inclinometer 20 and/or a plurality of accelerometers 22.

These sensors may be configured so as to span the 3D coordinate frame, that is, 3 degrees of freedom of linear motion plus 3 degrees of freedom of rotation. After appropriate initialization, the readings from the sensors may be integrated to produce angular attitude, velocity and position. If the integration is initialized before tracking the spine, then the position will indicate the location of the spine, while the angular orientation will reflect the spine angles.

The sensor probes 14 may comprise optical navigators such as an optical mouse sensor, for example, a microprocessor for the image processing and communication, a led for providing illumination and the required optics.

The inclination tracking device 12 may further comprise an operation switch, (Reset/Stop button) for controlling the measurement cycle, an LCD (or display) for presenting results to the user and a speaker for communicating commands to the operator.

In an embodiment of the invention, the inclinometer 10 may also be equipped with a base containing a rechargeable battery for providing power supply to the sensors and the CPU and a transmitter which may be Bluetooth enabled and may be used to upload/download data through an USB cable to an external computer, for example.

The inclination tracking device 12 comprises a substantially rectangular element 16 having an indentation or semi-circular arch 20 formed in the center of one edge of element 16. The arch is configured to pass over the spinal column. In one embodiment of the invention, the arch 20 may be approximately 3 cm in diameter. In an exemplary embodiment of the invention, the inclinometer 10 may be formed from non-warp, lightweight and hygienic material.

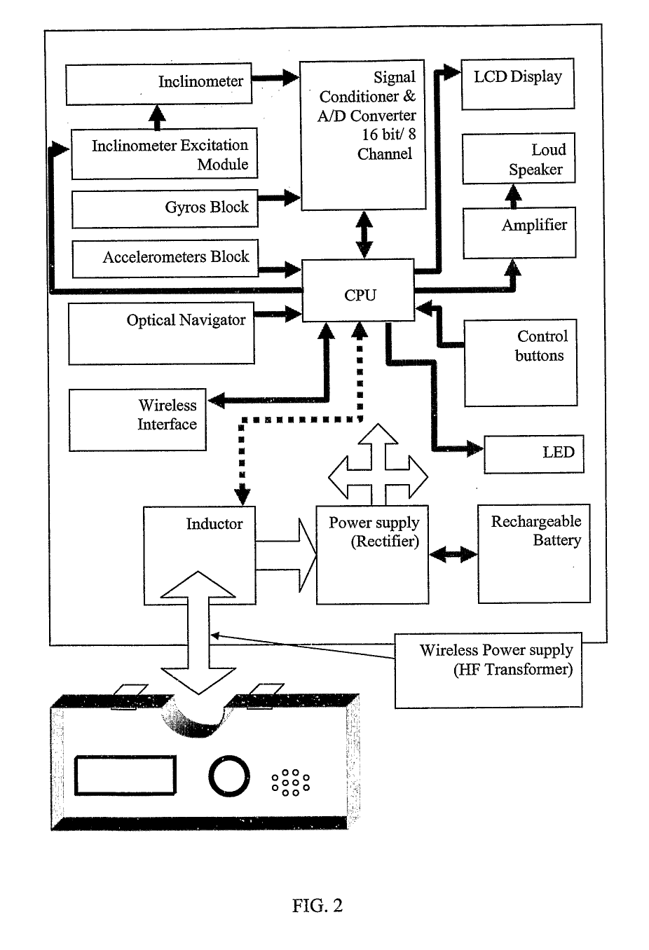

Reference is now made to FIG. 2, which is a schematic block diagram illustration showing the connectivity of the internal components of the inclination measuring device 10. The inclination measuring device 10 comprises a CPU (central processing unit) connected to the plurality of gyros 18, inclinometer 20 plurality of accelerometers 22, inclinometer excitation module, signal conditioner and A/D converter, optical navigator (sensor probes 14), LCD display, loudspeaker, amplifier, control buttons, LED, wireless interface and power supply. Optionally, an inductor may be connected to the power supply. The inductor may supply the connection for a wireless power supply (HF transformer, for example) for recharging the inclinometer 10.

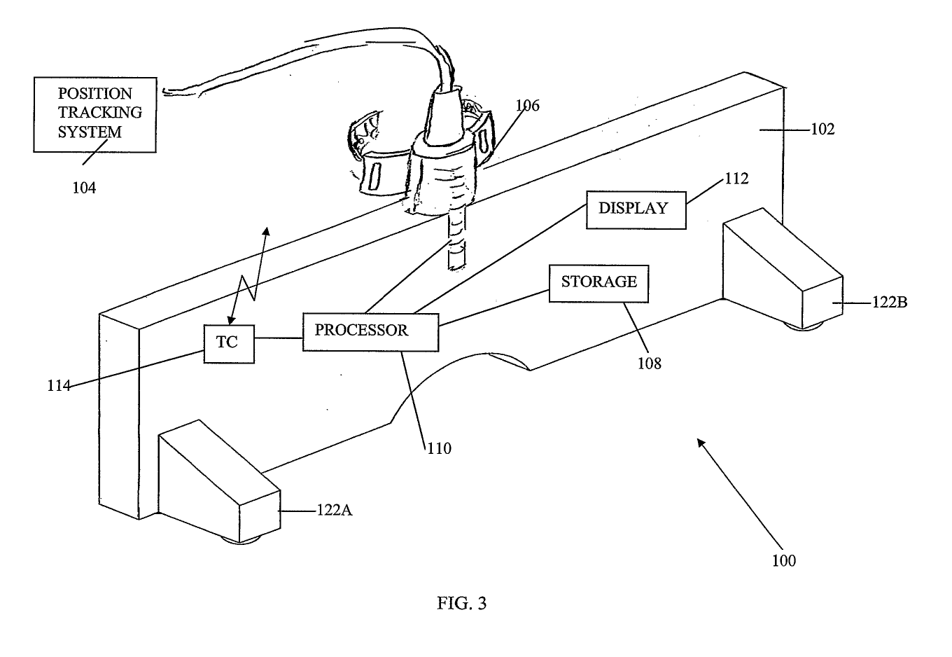

In a further embodiment of the invention, as shown in FIG. 3, to which reference is now made, the inclinometer 100 may comprise an inclination tracking device 102 which may be connected to an independent position tracking system 104 and an independent sensor probe 106. As will be appreciated by persons skilled in the art, the inclination tracking device 102 and sensor probe 106 may be adapted to incorporate the storage (108) and computing capabilities (110) such as a data processor, within the inclination tracking device 102 itself, which may also be fitted with a display screen (112). The inclination tracking device 102 may also be fitted with a transmitting device (114) for transmitting data to an external source.

The inclinometer 100 may comprise a pair of tracking devices 122a, 122b attached on either side of the arch 120, along the bottom edge of inclination tracking device 102. The tracking devices 122a, 122b provide stability and allow the inclination tracking device 102 to stay on track and glide over the back. In an exemplary embodiment of the invention, small wheels may be fitted to the tracking devices 22a, 22b.

The sensor probe 106 may be configured to be removable from the inclinometer 100. In an alternative embodiment of the invention, the sensor probe 106 may be configured to be attached to a person's finger.

The spinal curve is sensed in the described preferred embodiments by the sensor probe, which is fitted to a finger, sensing the spinous process of each vertebra.

In an alternative embodiment of the invention, sensor probe 106 may be in communication with a position sensor and tracking system 104. The position sensor and tracking system 104 may be of an electromagnetic field type, as described in U.S. Pat. No. 6,524,260, or any other similar device.

The sensor probe 106 is configured to measure the position and inclination of the inclination tracking device 104 as it is moved along an object, such as a spine, for example. Since the position of the sensor probe 106 is fixed in relation to the inclination tracking device 102, the inclinometer 100 does not need to be calibrated each time it is used.

The tracked positions may be recorded in an input/output storage device for further processing. In addition, the output of the workstation can be transmitted via a telecommunication device to a remote location, via a telephone line, for viewing, recording, or further processing, for example.

The inclinometer 10 (of FIG. 1) and the inclinometer 100 (of FIG. 3) may be used for preventive health screening and in the orthopedic field. By using the inclinometer as an accurate automatic or semi-automatic first-level screening of school-aged children for spinal deformities, such as scoliosis and kyphosis, for example, the school screening system can quickly and efficiently filter out children needing further testing.

Furthermore, in the orthopedic field, inclinometers 10 or 100 may be incorporated into the contour mapping system described in U.S. Pat. Nos. 6,500,131 and 6,524,260 (assigned to common assignees of the present application), to semi-automate the trunk rotation measurements as a part of the standard spine scan procedure.

In the exemplary application of this embodiment, the use of the inclinometer 10 of FIG. 1 for the measurement of the trunk inclination of a patient will now be described.

Measurement of Trunk Rotation

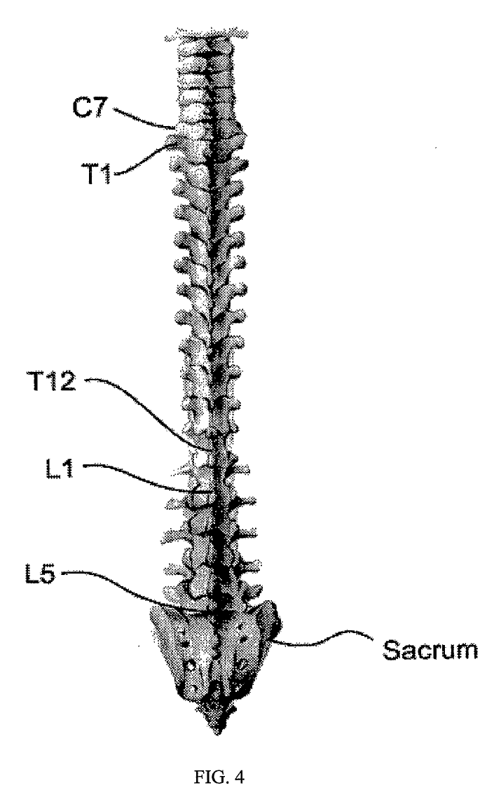

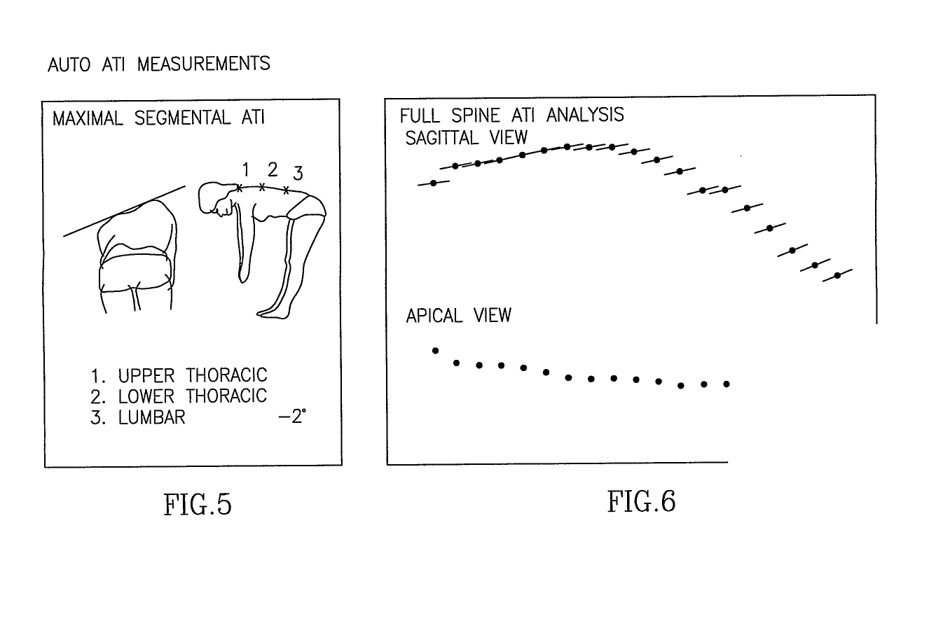

To measure the trunk rotation of a patient, the patient should hold out his arms perpendicular to his body with palms touching while standing. The patient should then bend over as far as possible (see FIG. 5), with the emitter approximately at the level of the naval. The patient's shoulders should be approximately at the hip level (if physically possible). Trunk rotation may be measured by holding the inclinometer 10 with one hand perpendicular to the back with the indented semi-circular arch 20 over the spinous process of C7. The inclinometer 10 should then be glided over the spine (following the spine contour as closely as possible) from C7 (see FIG. 4) down to S1 (start of the sacrum) The examiner should use his/her free hand to palpate gently down the spine and guide the inclinometer 10 over the spine contour.

The optical sensor probes 14 may be configured to take at least one reading per vertebrae and preferably a plurality of readings along the back contour. In an exemplary embodiment, the optical sensor probes 14 record 40 readings per second. The system is configured to output quantitative measurements (in degrees) of the maximal trunk rotation measurements in the upper thoracic, mid-thoracic, and lumbar regions. In addition the vertebral level of the trunk rotation measurements and the direction of the inclination (that is the difference in height between left and right may also be calculated.

Since the inclinometer 10 of the present invention uses optical sensor probes 14, the measurement of a patient's back is not dependent on the patient bending over into an almost horizontal position (the non-limiting example of FIG. 4) in order to obtain accurate readings. As will be appreciated by persons skilled in the art, the inclinometer is a digital device and is capable of measuring angular deviation irrespective of the position of object being measured. Thus, accurate results of trunk rotation, for example, may be obtained from a patient in any position, which is a major advantage over prior art devices. In an alternative embodiment of the invention, the inclinometer 10 may further comprise a sensor (not shown), such as an optical sensor, for example, for measuring the length of a person's spine. Any measurement technique, known in the art, such as the difference between x, y coordinates may be used to calculate the length of the spine. Similarly, the inclinometer of FIG. 3 may be used for carrying out these tests. In this case, the tracking devices 104 may be used in communication with the sensor 106.

FIG. 6 is an exemplary graphical illustration of some of the results available of the measurement of trunk rotation. As shown, the screen shows a graphical display of the Sagittal (top right) and Apical (bottom right) views for a full spine ATI (Angle of Trunk Inclination) analysis for the upper thoracic, lower thoracic and lumbar regions (marked 1, 2, 3, respectively).

The screen may also graphical display the X, Y, Z coordinates of the readings and a graphical Coronal views. The display may also highlight the maximum inclination and location of the vertebrae in each of the upper thoracic, lower thoracic and lumbar regions. In the example shown, the maximum inclination in the lumbar region of −2 degrees is indicated. It will be appreciated by persons skilled in the art that computer processing techniques allow for the processing of data and for output in any format, and is not limited by the example given.

In a further embodiment of the invention, the inclinometer or inclination measuring device may be configured to incorporate special markers. The markers may be used in conjunction with Optical 3D tracking systems (instead of a magnetic field generator of FIG. 1), such as infra red (IR) or other cameras, to identify and calculate the inclination angles.

The above examples and description have been provided only for the purpose of illustration, and are not intended to limit the invention in any way. As will be appreciated by the skilled person, the invention can be carried out in a great variety of ways, employing more than one technique from those described above, all without exceeding the scope of the invention.

1. An inclination measuring device comprising:
an inclination tracking device configured to pass over the object whose angle of inclination is to be mapped, said object having a plurality of elements; and
a sensor probe in communication with said inclination tracking device, said sensor probe configured to sense the position of each of said plurality of elements.

2. The inclination measuring device according to claim 1, wherein said object to be mapped is the spine of a person and said elements are vertebrae.

3. The inclination measuring device according to claim 1, wherein said sensor probe is fixed in relation to said inclination tracking device.

4. The inclination measuring device according to claim 1, wherein said sensor probe comprises optical sensors.

5. The inclination measuring device according to claim 1, wherein said sensor probe is configured to be removable from said inclination tracking device and is configured to be attachable to at least one finger of a user's hand.

6. The inclination measuring device according to claim 1, wherein said sensor probe further comprises a position sensor and tracking system in communication therewith.

7. The inclination measuring device according to claim 1, wherein said inclination tracking device comprises one of a group of devices for calculating the angles of inclination including gyroscopic inclinometer device, inclinometer, accelerometer, a magnetic field generator and Optical 3D tracking systems.

8. The inclination measuring device according to claim 1, wherein said inclination tracking device comprises a processing unit and at least one of a group of devices including a data storage device and a display screen in communication with said processing unit.

9. The inclination measuring device according to claim 8, wherein said inclination tracking device further comprises a transmitting device for transmitting data to an external source.

10. The inclination measuring device according to claim 8, wherein said inclination tracking device comprises an inductor in communication with said processing unit for supplying power via a wireless connection to a unit for recharging the inclination measuring device.

11. The inclination measuring device according to claim 2, wherein said sensor probe is configured to record at least one reading for each vertebrae.

12. The inclination measuring device according to claim 8, wherein said processing unit is programmed to record data including maximal trunk rotation measurements of at least one of group of vertebrae, including the upper thoracic, mid-thoracic, and lumbar regions of the spine.

13. The inclination measuring device according to claim 12, wherein said processing unit is programmed to compute and display the data showing at least one of a group including Coronal, Sagittal and Apical views of the spine.

14. The inclination measuring device according to claim 12, wherein said processing unit is programmed to compute and display the maximum inclination and/or location of the vertebrae in each of the upper thoracic, lower thoracic and lumbar regions of the spine.

15. The inclination measuring device according to claim 2, wherein said sensor probe is configured to record at least one of a group comprising the vertebral level of the trunk rotation measurements, the direction of inclination of each vertebrae, the difference in height between left and right of each vertebrae and the length of the spine.

16. The inclination measuring device according to claim 1, wherein said inclination measuring device is configured to measure the angular deviation irrespective of the position of object being measured.

17. The inclination measuring device according to claim 1, wherein said inclination tracking device comprises a substantially rectangular housing having an indentation formed in the center of one edge of said housing.

18. The inclination measuring device according to claim 17, wherein said inclination tracking device comprises a pair of tracking devices attached on either side of said indentation, along the bottom edge of rectangular said element.

19. The inclination measuring device according to claim 7, wherein said inclination tracking device comprises markers configured to be used in conjunction with said Optical 3D tracking systems to identify and calculate inclination angles of the vertebrae.
